(19) United States
(12) Patent Application Publication
(10) Pub. No.: US 2010/0267662 A1
(43) Pub. Date: Oct. 21, 2010

(54) AVENANTHRAMIDE-CONTAINING COMPOSITIONS

(75) Inventors: David A. Fielder, Edmonton (CA); Mark J. Redmond, Edmonton (CA); Ian W. Cottrell, Spring Hill, FL (US)

Correspondence Address: AKERMAN SENTERFITT, P.O. BOX 3188, WEST PALM BEACH, FL, 33402-3188, US

(73) Assignee: CEAPRO, INC., Edmonton, AB (CA)

(21) Appl. No.: 12/741,695

(22) PCT Filed: Nov. 6, 2008
(86) PCT No.: PCT/CA2008/002008

§ 371 Date: May 6, 2010

Related U.S. Application Data
(60) Provisional application No. 60/986,476, filed on Nov. 8, 2007.

(51) Int. Cl. A61K 31/196; A61K 31/716; A61P 29/00; A61P 17/00; A61P 33/14; A61P 17/04; A61P 33/10; A61P 33/00

(52) U.S. Cl. 514/54; 514/563

(57) ABSTRACT

Methods and compositions for treating or preventing a skin condition, an inflammation, an irritation or an allergy associated with an ectoparasitic infection or infestation on an animal. The methods involve applying to the skin of the animal a pharmaceutical composition that contains a therapeutically effective amount of one or more than one avenanthramide, an optional ecto and/or endo-parasiticidal agent, and a pharmaceutically acceptable diluent or carrier

### FIELD OF INVENTION

The present invention relates to pharmaceutical compositions for treating a skin disorder or condition or allergy in an animal. More particularly, the present invention relates to pharmaceutical compositions comprising a therapeutically effective amount of one or more than one avenanthramide, for treating a skin disorder or condition in an animal, and methods of using these compositions.

The present invention relates to the production and use of solubilised, liquid oat extracts or colloidal oatmeal with formulations having utility in the personal care, cosmetics, nutraceutical, and pharmaceutical industries. More specifically, the oat extract compositions or colloidal oatmeal of the present invention are useful as anti-irritants, anti-oxidants and skin-protection agents applied to the skin or when consumed.

### BACKGROUND OF THE INVENTION

Oats (Avena sativa), and especially colloidal oatmeal suspensions have been used historically as adjuncts to the treatment of atopic dermatitis. It is desirable to extract the active ingredients from the oat in order to facilitate the use of the grain in medicinal and cosmetic applications.

Oat derivatives such as colloidal oatmeal, hydrolysed oat protein, oat starch, and β glucan have been used in the cosmetics and pharmaceutical industries as a skin protectant which provides a smooth feel after use. Specifically, the carbohydrates and protein in the oat derivatives have been known to function as a protectant to aid in enhancing the skin's barrier properties and thereby soothe the skin. Oat β glucans and lipids have also been known to function as emollients to lubricate and soothe the skin. For example, colloidal oatmeal has been used for bar soaps, bath powders, lotions, and poultices to treat skin that has been damaged, irritated, or distressed by a wide variety of causes. However, some oat derivatives, for example, colloidal oatmeal, are not fully soluble in aqueous solutions and leave undesirable residues on the skin and other surfaces. U.S. Pat. No. 5,219,340 describes a cloth applicator designed to retain colloidal oatmeal insoluble fractions.

Furthermore, acid hydrolysed oat protein is known to have a strong odour which may adversely affect some consumer's acceptance of the product.

Liquid oat extracts prepared by extraction with alcohol, glycols, ethers, esters, mixtures, and aqueous mixtures thereof are typically unstable materials, which if not emulsified, readily separate into oil and aqueous phases which may further separate into soluble and insoluble phases. Alcohol soluble cereal proteins interact with a wide range of phenolic compounds naturally found in cereal grains, forming a chill haze or protein haze. These hazes will cause the extract to become turbid. Over time, the hazes will agglomerate resulting in an insoluble precipitate.

Paton (1995) Cosmetics and Toiletries 110:63 describes the cosmetic use of oat extracts and provides information on cosmetic formulations. The oat extract described, OSTAR ARRIVEEN™, is produced from oats by a pearling process by which oat bran is obtained, which was then extracted with solvent. Charcoal was used in the process to clarify the preparation. The product is typically a dark brown coloured, non-homogeneous, bi-phasic extract. The utility of this product was limited by instability resulting in varying performance. The product could not be sterilised resulting in a high microbial load due to non-kilned, non-stabilised oat bran. This oat extract was purported to have anti-erythemal properties, however, the active ingredients were not identified.

Collins et al. (U.S. Pat. No. 5,169,660) describes the preparation of bran from cereal grains using aqueous alcohol extraction (83% w/w) and the recovery of crude by-products from waste through ion-exchange chromatography. The described process does not use pH pre-treatment or membrane filtration and so results in only recovered small quantities of by-product from waste. Utility is not described in cosmetic applications and pharmaceutical claims are not enabled. Furthermore, the Ion Exchange Chromatographic process described in Collins et al. may degrade some avenanthramides, thereby reducing the overall percent recovery of these compounds.

Collins in Oats: Chemistry and technology (1986) Ed. Webster AACC St. Paul, Minn. pp 227-286 describes oat phenolic compound structure, occurrence and phytological function. Methods of extraction of these compounds and potential utility in the cosmetic and medical fields of use were not disclosed.

Onitsuka et al. (U.S. Pat. No. 5,716,605) describe the use of glycolic extracts of oats for the treatment and care of hair and the scalp. The extraction method described is different to the method described in the present invention.

Cioca et al. (U.S. Pat. No. 5,552,135) describes improved sunscreen compositions including extracts from cereal plants. The primary extraction is made with chloroform or ethanol and further processed in alcohol following evaporative concentration.

Hammonds et al. (PCT/US97/10724) describes fibrous sheet materials containing oat extracts to provide a soothing effect to the skin of the user. The oat extracts claimed are made by treating oats with extraction agents by methods known to those skilled in the art. Methods of preparing oat extracts are not disclosed; the described product used specific concentrations of OSTAR ARRIVEEN™ in the preferred mode.

Zimmerman (U.S. Pat. No. 5,888,521) describes compositions for topical use consisting of hydroxycarboxylic acid and oat extract, and also relates to methods of enhancing the rate of skin desquamation. Methods of preparing oat extracts are not disclosed; the described product used specific concentrations of OSTAR ARRIVEEN™ in the preferred mode.

Roger et al. (U.S. Pat. No. 5,026,548) describes a phospholipid surfactant for use as a viscosity reducing agent in chocolate, or an emulsifier, surfactant or foam stabilizer in the food and other industries, which is produced by extracting oats using an alcohol such as ethanol or propanol, extracting the alcohol extract with methanol and evaporating the methanol.

Targan (U.S. Pat. No. 5,468,491) describes a method for producing an aqueous oat syrup involving enzymatic digestion, cooking, filtration through on oat bed, and concentration to produce an extract composed of 80% sugars and 20% water. Utility is expressed as a flavour, colour, sweetener, and or texture enhancer. The composition is different from the liquid oat extract described in the present application.

Rouanet et al. (PCT/FR98/00826) describes a method for making a solid preparation of white colloidal oats, comprising the following steps: using cultivated oat seeds; stabilizing by at least one operation whereby dry vapour is injected followed by sudden cooling, preferably at about room temperature; pinning and drying; breaking and eliminating the bran; dimensional selecting of particles.

Vallet Mas et al. (EP 0 661 047) describes die combination of topical anti-histamines with solid oat flour to form an emulsion for the treatment of itching, reduction of inflammation and facilitation of spreading over the effected area. No reference is made to the anti-irritant potential of oat extracts.

Kovacs (EP 0 282 002) describes the use of combinations of nettle (Urtica) and oat extracts as food additives or pharmaceutical preparations. The methods of preparing the oat extracts are described as, “classical methods” and no enabling details are provided.

Lawrence (U.S. Pat. No. 5,573,785) describes an oat derived, skin conditioning, cosmetic component produced by dispersing in water a water-soluble fibre composed of about 4 to 6 weight percent beta glucan, about 1 to 5 weight percent fat, about 80 to 94 weight percent carbohydrates and less than 8 weight percent protein. No data relating to anti-irritant and redness reduction is provided.

### SUMMARY OF THE INVENTION

The present invention relates to pharmaceutical compositions for treating a skin disorder or condition or allergy in an animal. More particularly, the present invention relates to pharmaceutical compositions comprising a therapeutically effective amount of one or more than one avenanthramide, for treating a skin disorder or condition in an animal, and methods of using these compositions.

In a first aspect, the present invention provides a method for treating or preventing a skin condition (such as erythema or pruritus), an inflammation (e.g. otitis), an irritation or an allergy associated, for example, with an ectoparasitic infection or infestation on an animal, comprising applying to the skin of the animal a therapeutic pharmaceutical composition comprising a therapeutically effective amount of one or more than one avenanthramide and a pharmaceutically acceptable diluent or carrier.

In a second aspect, the present invention provides a method for treating or preventing a skin condition (such as erythema or pruritus), an inflammation (e.g. otitis), an irritation or an allergy associated, for example, with an ectoparasitic infection or infestation on an animal, comprising applying to the skin of the animal a therapeutic/parasiticidal pharmaceutical composition comprising a therapeutically effective amount of one or more than one avenanthramide, a therapeutically effective amount of an ectoparasiticidal agent (e.g. an insecticide) and a pharmaceutically acceptable diluent or carrier.

In a third aspect, the present invention provides a method for treating or preventing a skin condition (such as erythema or pruritus), an inflammation (e.g. otitis), an irritation or an allergy associated, for example, with an ectoparasitic infection or infestation on an animal and an endoparasitic infection or infestation in the animal, comprising applying to the skin of the animal a therapeutic/parasiticidal pharmaceutical composition comprising a therapeutically effective amount of one or more than one avenanthramide, a therapeutically affective amount of an ectoparasiticidal agent (e.g. an insecticide), a therapeutically effective amount of an endoparasiticidal agent (such as an anti-helminthic agent) and a pharmaceutically acceptable diluent or carrier.

In a fourth aspect, the present invention provides a therapeutic pharmaceutical composition for treating or preventing a skin condition (such as erythema or pruritus), an inflammation (e.g. otitis), an irritation or an allergy associated, for example, with an ectoparasitic infection or infestation on an animal, comprising a therapeutically effective amount of one or more than one avenanthramide and a pharmaceutically acceptable diluent or carrier.

In a fifth aspect, the present invention provides a therapeutic/parasiticidal pharmaceutical composition for treating or preventing a skin condition (such as erythema or pruritus), an inflammation (e.g. otitis), an irritation or an allergy associated, for example, with an ectoparasitic infection or infestation on an animal, comprising a therapeutically effective amount of one or more than one avenanthramide, a therapeutically effective amount of an ectoparasiticidal agent (e.g. an insecticide) and a pharmaceutically acceptable diluent or carrier.

In a sixth aspect, the present invention provides a therapeutic/parasiticidal pharmaceutical composition for treating or preventing a skin condition (such as erythema or pruritus), an inflammation (e.g. otitis), an irritation or an allergy associated, for example, with an ectoparasitic infection or infestation on an animal and an endoparasitic infection or infestation in the animal, comprising a therapeutically effective amount of one or more than one avenanthramide, a therapeutically affective amount of an ectoparasiticidal agent (e.g. an insecticide), a therapeutically effective amount of endoparasiticidal agent (such as an anti-helminthic agent) and a pharmaceutically acceptable diluent or carrier.

In an example of the above-defined methods and pharmaceutical compositions, the allergy associated with an ectoparasitic infection or infestation is flea allergy dermatitis.

In another example, the pharmaceutical compositions of the present invention may further comprise a therapeutically effective amount of a cereal β-glucan and/or one or more than one other anti-inflammatory agent.

The one, or more than one avanenathramide of the pharmaceutical compositions defined above may be produced by a process comprising the following steps:

a. Milling whole oats,

b. Extracting the resulting oatmeal or milled oat fraction with a solvent,

c. Adjusting the pH of the resulting oat extract to <4.0,

d. Membrane filtration of the oat extract with a pH <4.0 through a membrane <10⁴ MWCO.

In further examples, the one, or more than one avenanthramide may be present in the pharmaceutical compositions of the present invention at a concentration of from about 0.0001 to about 375 ppm or any value or subrange therebetween, 0.001 to about 375 ppm or any value or subrange therebetween, from about 0.0001 to about 150 ppm or any value or subrange therebetween, from about 0.001 to about 150 ppm or any value or subrange therebetween, from about 0.01 to about 150 ppm or any value or subrange therebetween, from about 0.01 to about 50 ppm or any value or subrange therebetween, from about 0.3 to about 15 ppm or any value or subrange therebetween, or from about 1.5 to about 4.5 ppm or any value or subrange therebetween.

The pharmaceutical compositions of the present invention may comprise from about 0.1 to about 25 weight percent or any value or subrange therebetween, or from about 1 to about 10 weight percent or any value or subrange therebetween, of an oat extract comprising the one, or more than one avenathramide at a concentration of from about 1 to about 1500 ppm or any value or subrange therebetween, or from about 3 to about 450 ppm or any value or subrange therebetween, based on the oat extract.

The amount of cereal 3 glucan in the pharmaceutical compositions of the present invention may be from about 0.001 wt % to about 1 wt % or any value or subrange therebetween, from about 0.01 wt % to about 0.8 wt % or any value or subrange therebetween, or from about 0.1 wt % to about 0.5 wt % or any value or subrange therebetween.

In addition, the amount of the ecto- and/or endo-parasiticidal agents present in the therapeutic/parasticidal compositions of the present invention may be from about 0.001 wt % to about 20 wt % or any value or subrange therebetween, from about 2 wt % to about 15 wt % or any value or subrange therebetween, or from about 5 wt % to about 10 wt % or any value or subrange therebetween.

The therapeutic/parasticidal agents of the present invention are advantageous in that they can simultaneously treat the symptoms of an ectoparastic infection or infestation on the skin of an animal and kill or control the ectoparasites, which are responsible for causing the symptoms.

### DETAILED DESCRIPTION OF THE INVENTION

The present invention relates to pharmaceutical compositions for treating a skin disorder or condition or allergy in an animal. More particularly, the present invention relates to pharmaceutical compositions comprising a therapeutically effective amount of one or more than one avenanthramide, for treating a skin disorder or condition in an animal, and methods of using these compositions.

The practice of the present invention will employ, unless otherwise indicated, conventional methods of chemistry, cereal chemistry, cosmetic chemistry, pharmacy, and biochemistry within the skill of the art.

All publications, patents and patent applications cited herein, whether supra or infra, are incorporated by reference in their entirety.

As used in this specification and the appended claims, the singular forms “a”, “an”, and “the” include the plural references unless the content clearly indicates otherwise. Thus the term “an avenanthramide” can include more than one member of the group of avenanthramides.

Definitions

In describing the present invention, the following terms are employed, and are intended to be defined as indicated below.

By an “avenanthramide” in singular or plural is meant a member of a group of more than 40 naturally occurring anthranilic acid derivatives found in oats (Collins. J. Agric. Food Chem. 37: 60-66 (1989)), carnations and butterfly eggs, and are unique to cereal grains, or a synthetically produced avenanthramide as described in United States Patent Application Publication No. 2006/0089413, or a synthetically produced derivative of an avenanthramide as described, for example, in WO2006/087393, the disclosures of which is incorporated herein by reference. Methods of synthesis of avenanthramides are described in U.S. Pat. Nos. 6,096,770 and 6,127,392 as well as Japanese Patent No. J60019-754-A and Hungarian Patent HU 200 996 B, the disclosures of which are incorporated herein by reference. Nomenclature follows the convention described in Oats: Chemistry and technology (1986) Ed. Webster AACC St. Paul. Minn. pp 227-286 with specific avenanthramide compounds represented by the prefix ‘AF’ followed by a number, for example AF-1, AF-2 and AF-6 as illustrated below.

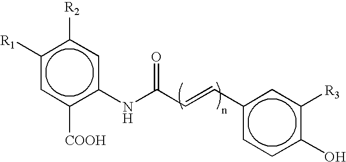

| Name | R₁ | R₂ | R₃ |
|---|---|---|---|
| AF-1 | OH | H | H |
| AF-2 | OH | H | OCH₃ |
| AF-6 | OH | H | OH |

By “Oatmeal” is meant the product of grinding or milling whole naked (hulless) oats or oat groats.

By “Oat bran” is meant the product of grinding oat groats or rolled oats and separating the resulting oatmeal by sieving, bolting and/or other suitable means into fractions such that the oat bran fraction is not more than 50% of the starting material, and has a total glucan content of at least 5.5% (dry weight basis) and a total dietary fibre content of at least 16.0%.

By “Oat flour” is meant the product of grinding oat groats or rolled oats and separating the resulting oatmeal by sieving, bolting and/or other suitable means into flour fractions, which completely pass through a 100 Mesh screen.

By “Ultra-filtration (UF)” is meant the process of tangential filtration whereby solutes are retained by a membrane the parameters of which are based on molecular weight.

By “Reverse Osmosis (RO)” is meant the process of tangential filtration whereby water and/or low molecular weight solvent, for example ethanol, passes through a membrane thereby concentrating the Retentate.

By “Membrane filtration” (MF) is meant the process of filtration whereby solutes are retained by a membrane, the parameters of which are based on molecular weight. UF and RO are examples of MF.

By “Molecular Weight Cut-Off (MWCO)” is meant that above a specified MWCO, the membrane will retain most species of that molecular weight.

By “Permeate” is meant the fluid containing the solutes that passes through the UF/RO membrane.

By “Retentate” is meant the fluid containing the solutes that are retained by the UF/RO membrane.

By “Flow” is meant the volumetric filtration rate (flow rate) through a given membrane area per unit time. Units are usually litres per square meter per hour (LMH).

By “Diafiltration” is meant the efficient method of recovering solutes (<MWCO) in low concentrations from the solution, by addition of fresh solvent at a rate equal to the UF rate. At constant volume, the permeate solutes are removed from the Retentate. The rate of recovery is a function of the UF rate and is independent of the concentration of the permeate solutes.

By “Membrane fouling” or “concentration polarization” is meant the accumulation of retained or absorbed material on the membrane surface.

By “Concentration” is meant the accumulation of rejected permeate solutes on the membrane

By “Percent recovery” is meant the amount of desired solute as a percentage of the amount present in the feed-stream.

By “cereal” is meant any of several grains such as, but not limited to, cultivars of barley, oat, wheat, rye, sorghum, millet, and corn.

By “glucan” is meant a homopolysaccharide consisting only of glucose.

By “cereal β-glucan” is meant a glucan with a β(1-3)-linked glucopyranosyl backbone, or a β(1-4)-linked glucopyranosyl backbone, or a mixed β(1-3) β(1-4)-linked glucopyranosyl backbone, which is derived from a cereal source.

By “animal” is meant an animal susceptible to infection or infestation by an ecto- or an endoparasite, for example, an animal such as a cat, a dog, sheep, a goat, a cow, or a human; or a bird.

By “ectoparasitic infection or infestation” is meant an infection or infestation caused by an organism that lives on or in the skin of an animal, such as an organism from the suborder of Anoplura, e.g. Haematopinus spp., Linognathus spp., Solenopotes spp., Pediculus spp., Pthirus spp.; from the order of Mallophaga, e.g. Trimenopon spp., Menopon spp., Eomenacanthus spp., Menacanthus spp., Trichodectes spp., Felicola spp., Damalinea spp., Bovicola spp; from the order of the Diptera e.g. Chrysops spp., Tabanus spp., Musca spp., Hydrotaea spp., Muscina spp., Haematobosca spp., Haematobia spp., Stomoxys spp., Fannia spp., Glossina spp., Lucilia spp., Calliphora spp., Auchmeromyia spp., Cordylobia spp., Cochliomyia spp., Chrysomyia spp., Sarcophaga spp., Wohlfartia spp., Gasterophilus spp., Oesteromyia spp., Oedemagena spp., Hypoderma spp., Oestuus spp., Rhinoestrus spp., Melophagus spp., Hippobosca spp., or from the order of Siphonaptera e.g. Ctenocephalides spp., Echidnophaga spp., Ceratophyllus spp. Particular examples include fleas (Ctenocephalides felis, Ctenocephalides canis, Ctenocephalides sp. and the like), ticks (Rhipicephalus sp., Ixodes sp., Dermacentor sp., Amblyoma sp., Haemaphysalis longiconis and Boophilus microplus and the like), mites (Demodex sp., Sarcoptes sp., Otodectes sp. and the like), lice (Trichodectes sp., Cheyletiella sp., Lignonathus sp., and the like), mosquitoes (Aedes sp., Culux sp., Culex pipiens, Anopheles sp., and the like) and flies (Hematobia sp., Haematobia irritans, Musca sp., Musca domestica, Musca hervei, Musca bezzi, Stomoxys sp., Dematobia sp., Coclyomia sp., and the like).

By “endoparasitic infection or infestation” is meant an infection or infestation caused by an organism living inside a tissue or bloodstream of an animal, such as roundworms (e.g., Toxocara canis, Toxascaris leonine); tapeworms (e.g., Dipylidium caninum, Taenia piriformis, Echinococcus granulosus, E. multilocularis); whipworms (e.g., Trichuris vulpis, T. campanula, T serrata); hookworms (e.g., Ancylostoma caninum, A. braziliense, A. tubaeforme, Uncinaria stenocephala); heartworms (e.g. Difilaria immitis); stomach worms (e.g. Physaloptera spp.) and microscopic parasites (e.g. Coccidia, Giardia and Strongloides spp.)

The present invention provides both therapeutic and therapeutic/parasiticidal compositions for treating or preventing a skin condition, an inflammation, an irritation or an allergy associated with an ectoparasitic infection or infestation on an animal.

In particular, the present invention provides a therapeutic pharmaceutical composition for treating or preventing a skin condition (such as erythema or pruritus), an inflammation (e.g. otitis), an irritation or an allergy associated with an ectoparasitic infection or infestation on an animal, comprising a therapeutically effective amount of one or more than one avenanthramide, a therapeutically effective amount of an optional ectoparasiticidal agent, and a pharmaceutically acceptable diluent or carrier.

In addition, the present invention provides a therapeutic/parasiticidal pharmaceutical composition for treating or preventing a skin condition (such as erythema or pruritus), an inflammation (e.g. otitis), an irritation or an allergy associated with an ectoparasitic infection or infestation on an animal and an endoparasitic infection or infestation in the animal, comprising a therapeutically effective amount of one or more than one avenanthramide, a therapeutically affective amount of an ectoparasiticidal agent, a therapeutically effective amount of endoparasiticidal agent (such as an anti-helminthic agent) and a pharmaceutically acceptable diluent or carrier.

The pharmaceutical compositions of the present invention may be applied to an animal in the form of a foaming shampoo, a dip, an aerosol spray, a pump spray, a lotion, a concentrated solution (for use, for example, as a spot-on treatment), a dilute solution (for use, for example, as a spray-on treatment), a gel, lotion, ointment, a cream, an oil-in-water or water-in-oil emulsion, a suspension, a powder or in any other form suitable for applying as a topical composition to an animal. In addition, the pharmaceutical compositions may be applied to the animal dropwise, or brushed on, poured on, spotted on, rubbed in, sprayed on, splashed on, or applied by dipping or bathing.

In a particular example, the pharmaceutical composition of the present invention may be applied by pouring, dripping or spotting the composition to a small area of the skin or fur of the animal, for example, at the base of the skull, on the neck, between the shoulder blades or on the back of the animal. After being applied, the components of the composition can then translocate or spread out across the fur and/or skin of the animal to provide a broad coverage on the surface of the skin of the animal, and deliver the avenanthramide, and any parasiticidal agents and cereal β glucan present in the composition to an affected site on the skin of the animal. The composition can be applied prior to the onset of an adverse skin condition or after such a condition becomes apparent.

If the composition is applied by pouring or spotting to a confined area of the fur or skin of the animal, it may contain carriers, for example spreading oils, to aid in the distribution of the avenanathramides and any other active components present in the composition on the surface of the skin of the animal. Examples of suitable carriers include, without limitation, physiologically acceptable vegetable or synthetic oils (e.g., olive oil, groundnut oil, sesame oil, pine oil, linseed oil or castor oil), dipropylene glycol pelargonate, paraffin oils, silicone oils, oily solutions; alcoholic and isopropanolic solutions, for example solutions of 2-octyldodecanol or oleyl alcohol; solutions of esters of monocarboxylic acids, such as isopropyl myristate, isopropyl palmitate, lauric acid oxal ester, oleyl oleate, decyl oleate, hexyl laurate, capric acid esters of saturated fatty alcohols with a chain length of C₁₂-C₁₈; solutions of esters of dicarboxylic acids, such as dibutyl phthalate, diisopropyl isophthalate, diisopropyl adipate or di(n-butyl)adipate, or also solutions of esters of aliphatic acids, e.g. glycols, solutions of triglycerides.

It has been demonstrated (Internal study: “A double blind Clinical Evaluation of the Efficacy of Two Oat-protein Shampoos” by Pukay, B. P., Baker, B., Hannigan, M., and Purcell, T.) that an application of 5-13.3 ng/cm² of avenanthramides to the skin of animals will provide relief from skin conditions such as pruritis and other dermatological problems.

The following table lists the volume of a therapeutic or therapeutic/parasiticidal pharmaceutical composition containing 5, 10 or 20 ppm of avenanthramides (AV) that should be applied to the skin of a dog in order to achieve a coverage of 5 ng AV/cm² or 13.3 ng AV/cm² of skin surface.

| | | Amount of AV Applied (mg) | | | |
|---|---|---|---|---|---|
| | Surface Area | to achieve a coverage of: | Volume of 5 ppm AV | Volume of 10 ppm AV | Volume of 20 ppm AV |
| Weight of | of Skin of | 5 ng AV/cm² (A) or | Liquid Composition | Liquid Composition | Liquid Composition |
| Dog (kg) | Dog (cm²) | 13.3 ng AV/cm² (B) | Applied (mL) | Applied (mL) | Applied (mL) |
| 5 | 2900 | 0.0145 (A) | 2.9 | 1.4 | 0.7 |
| | | 0.0386 (B) | 7.7 | 3.8 | 1.9 |
| 11 | 4900 | 0.0245 (A) | 4.9 | 2.4 | 1.2 |
| | | 0.0652 (B) | 13.0 | 6.6 | 3.3 |
| 22 | 7800 | 0.0390 (A) | 7.8 | 3.9 | 2.0 |
| | | 0.1022 (B) | 20.4 | 10.2 | 5.1 |
| 44 | 12500 | 0.0625 (A) | 12.5 | 6.2 | 3.1 |
| | | 0.1669 (B) | 33.4 | 16.6 | 8.3 |

As illustrated above, the volume of the liquid composition that is applied can be easily changed by adjusting the concentration of the avenanthramides in the composition. The listed volumes for a 5 ppm AV liquid composition are similar to the recommended applied volumes for commercially available topical insecticidal compositions for dogs of similar weight.

For a spray application, the concentration of avenanthramides in the therapeutic or therapeutic/parasiticidal pharmaceutical compositions of the present invention should be reduced so that the appropriate amount of the composition can be applied from a sprayer (e.g. a pump or aerosol) onto the affected area on the animal's skin. For example, if the area affected on the skin of a 5 kg canine is 10×10 cm², the volume of the therapeutic or therapeutic/parasiticidal pharmaceutical composition that should be applied is 0.1 ml of a 5 ppm AV solution to provide 0.0005 mg of AV. This volume (0.1 mL) is too small a volume to be effectively applied by spraying. As a result, this volume should be diluted to a larger volume, for example 5 mL to produce a composition having an avevnanthramide concentration of 0.1 ppm, so that the composition can be more easily applied over the entire affected area of the skin of the animal. For topical applications (e.g. spot-on treatments), a higher concentration of avenanthramides should be present in the pharmaceutical compositions (for example, 10-20 ppm).

The therapeutic composition of the present invention may be prepared by diluting an oat extract or colloidal oatmeal containing one, or more than one avenanthramide, such as the oat extract or colloidal oatmeal described in the present application, or a commercially available oat extract or colloidal oatmeal (for example, Ceapro Inc.'s 100 ppm avenanthramide extract), or one, or more than one isolated natural or synthetic avenanthramide with a pharmaceutically acceptable diluent and/or carrier to form a solution, suspension or emulsion.

The therapeutic/parasiticidal compositions of the present invention may generally be prepared by adding a composition, oat extract, oat extract concentrate or colloidal oatmeal, which contains one or more avenanthramides (such as the therapeutic composition, oat extract or colloidal oatmeal described in the present application), to a commercially available parasiticidal composition, which contains one or more than one ectoparasiticidal agent and/or one or more than one endoparasiticidal agent. If the avenanthramide-containing composition, or the oat extract, the oat extract concentrate or the colloidal oatmeal containing one or more avenanthramides is water-based, then it may be necessary to reduce it to a residue or lyophilize it to a powder to permit the one or more than one avenanthramide contained within it to be completely solubilized with the commercially available parasiticidal composition.

Alternatively, the therapeutic/parasiticidal composition may be prepared by combining one or more than one avenanthramide (either in the form of an isolated product, or as an oat extract, a concentrated oat extract, a colloidal oatmeal or a dried, lyophilized or volume-reduced form thereof) with one or more than one ectoparasiticidal agent and/or one or more than one endoparasiticidal agent and a suitable diluent (e.g. solvent) and/or carrier to form a solution, suspension or emulsion. If the avenanthramide-containing composition, or the oat extract, the oat extract concentrate or the colloidal oatmeal containing the one or more than one avenanthramide is water-based, then it may be necessary to reduce it to a residue or lyophilize it to a powder to permit the one or more than one avenanthramide to be completely solubilized within the solvent used to dissolve the one or more than one ectoparasiticidal agent and/or one or more than one endoparasiticidal agent.

Additional components that may be added to the therapeutic or therapeutic/parasiticidal pharmaceutical compositions of the present invention, include, without limitation, a solubilizer, for example, polyvinylpyrrolidone, polyoxyethylated castor oil, or polyoxyethylated sorbitan esters; an acid; a base; a buffer salt; an antioxidant; a preservative such as benzyl alcohol, trichlorobutanol, p-hydroxybenzoic esters, n-butanol; a perfume; a colorant; a surfactant; a wetting agent; a light stabilizer and a tackifier such as a cellulose derivative, a starch derivative, a polyacrylate, or a natural polymer such as an alginate or gelatin.

The therapeutic/parasiticidal composition of the present invention can be prepared in the form of a gel by adding a thickener to a solution prepared in the manner described above. Examples of suitable thickeners include without limitation inorganic thickeners such as bentonites, colloidal silica, or aluminium monostearate; or organic thickeners such as cellulose derivatives, polyvinyl alcohols and their copolymers, acrylates and methacrylates.

The therapeutic/parasiticidal compositions of the present invention may be prepared in the form of an emulsion by dissolving one or more avenanthramides and one or more than one ectoparasiticidal agent and/or one or more than one endoparasiticidal agent in one of a hydrophobic phase and a hydrophilic phase and homogenizing this phase with a solvent of the other of the hydrophobic phase and a hydrophilic phase, in the presence of a suitable emulsifier and, optionally, other adjuvants such as colorants; absorption accelerators; preservatives; antioxidants such as potassium metabisulphite, ascorbic acid, butylhydroxytoluene, butylhydroxyanisole or tocopherol; light stabilizers such a benzophenone or novantisolic acid; and viscosity-increasing substances and substances which stabilize an emulsion, such as carboxymethylcellulose, methylcellulose and other cellulose and starch derivatives, polyacrylates, alginates, gelatin, gum arabic, polyvinylpyrrolidone, polyvinyl alcohol, copolymers of methyl vinyl ether and maleic anhydride, polyethylene glycols, waxes, colloidal silica, or mixtures of the substances mentioned.

Nonlimiting examples of ectoparasiticidal agents that may be used in the therapeutic/parasiticidal compositions of the present invention include fipronil, imidacloprid, permethrin (canines only), phenothrin, dinotefuran, acetamiprid, and metaflumizone. Juvenile hormone mimics such as methoprene or pyriproxyfen can also be included to kill flea eggs. Specific examples of parasiticidal compositions that may be used in conjunction or combined with the therapeutic compositions of the present invention comprising one or more avenanthramides include, without limitation, those described in U.S. Pat. Nos. 7,271,184; 7,132,448; 6,998,131; 6,962,713; 6,933,318; 6,896,891; 6,759,407; 6,716,442; 6,685,954; 6,613,783; 6,538,013; 6,495,573; 6,482,425; 6,429,206; 6,426,333; 6,329,374; 6,232,328; 6,001,858; 6,096,329; 5,612,047; and 4,395,407, the disclosures of which are incorporated herein by reference.

Non-limiting examples of endoparasitic agents include moxidectin, praziquantel, pyrantel pamoate, fenbendazole, febantel, milbemycin oxime, emodepside, ivermectin, selamectin, and doramectin (the last four are in the class of macrocyclic lactones).

U.S. Patent Application Publication No.2006/0062817, the disclosure of which is incorporated by reference herein, describes compositions and combinations of ecto- and endoparasiticidal agents, which may used as part or in conjunction with the therapeutic compositions of the present invention.

The avenanthramides, the ectoparasiticidal agents and the ectoparasiticidal agents described above may alternatively be provided as a combination of separate compositions, which are administered separately, sequentially or simultaneously.

Accordingly, the present invention also provides a therapeutic/parasiticidal pharmaceutical combination for treating or preventing a skin condition (such as erythema or pruritus), an inflammation (e.g. otitis), an irritation or an allergy associated with an ectoparasitic infection or infestation on an animal, comprising a first pharmaceutical composition comprising a therapeutically effective amount of one or more than one avenanthramide and a pharmaceutically acceptable diluent or carrier, and a second pharmaceutical composition comprising a therapeutically affective amount of an ectoparasiticidal agent and a pharmaceutically acceptable diluent or carrier, wherein the first and second pharmaceutical compositions are for separate, sequential or simultaneous administration.

The present invention also provides a therapeutic/parasiticidal pharmaceutical combination for treating or preventing a skin condition (such as erythema or pruritus), an inflammation (e.g. otitis), an irritation or an allergy associated with an ectoparasitic infection or infestation on an animal and an endoparasitic infection or infestation in the animal, comprising a first pharmaceutical composition comprising a therapeutically effective amount of one or more than one avenanthramide and a pharmaceutically acceptable diluent or carrier, a second pharmaceutical composition comprising a therapeutically affective amount of an ectoparasiticidal agent and a pharmaceutically acceptable diluent or carrier, and a third pharmaceutical composition comprising a therapeutically effective amount of endoparasiticidal agent (such as an anti-helminthic agent) and a pharmaceutically acceptable diluent or carrier, wherein the first, second and third pharmaceutical compositions are for separate, sequential or simultaneous administration.

The packaging described in incorporated U.S. Patent Application Publication No. 2006/0062817 for holding compositions of different active ingredients may also be used to hold the separate pharmaceutical compositions of the combinations of the present invention.

Examples of solvents that may be used in the compositions of the present invention include without limitation: water; alkanols; glycols, such as propylene glycol; polyethylene glycols; polypropylene glycols; glycerol; aliphatic alcohols such as ethanol and butanol; aromatic alcohols such as benzyl alcohol, phenylethanol, phenoxyethanol; esters such as ethyl acetate, ethyl lactate, butyl acetate, benzyl benzoate; ethers such as alkylene glycol alkyl ethers, such as diethylene glycol monoethyl ether, dipropylene glycol monomethyl ether, and diethylene glycol mono-butyl ether; ketones such as acetone, or methyl ethyl ketone; aromatic and/or aliphatic hydrocarbons; vegetable or synthetic oils; DMF; dimethylacetamide; N-methylpyrrolidone; or 2-dimethyl-4-oxy-methylene-1,3-dioxolane, and mixtures thereof. Typical solvent systems for insecticide and/or pesticide-containing compositions according to the present invention, which can provide good translocation include ethyl lactate, benzyl alcohol, ethanol, diethylene glycol monoethyl ether, propylene carbonate and mixtures thereof.

Examples of a hydrophobic phase used in preparing emulsions include without limitation paraffin oils, silicone oils, natural vegetable oils such as sesame seed oil, almond oil, castor oil, synthetic triglycerides such as caprylic/capric acid biglyceride, triglyceride mixture with vegetable fatty acids of chain length C₈₋₁₂ or with other specifically selected natural fatty acids, partial glyceride mixtures of saturated or unsaturated fatty acids which may also contain hydroxyl groups, and mono- and diglycerides of the C₈/C₁₀-fatty acids, fatty acid esters such as ethyl stearate, di-n-butyryl adipate, hexyl laurate, dipropylene glycol pelargonate, esters of a branched fatty acid of medium chain length with saturated fatty alcohols of chain length C₁₆-C₁₈, isopropyl myristate, isopropyl palmitate, caprylic/capric esters of saturated fatty alcohols of chain length C₁₂-C₁₈, isopropyl stearate, oleyl oleate, decyl oleate, ethyl oleate, ethyl lactate, waxy fatty acid esters such as dibutyl phthalate, diisopropyl adipate, ester mixtures related to the latter, and other fatty alcohols such as isotridecyl alcohol, 2-octyldodecanol, cetylstearyl alcohol and oleyl alcohol, and fatty acids such as, for example, oleic acid and its mixtures.

Nonlimiting examples of the hydrophilic phase that may be used to prepare emulsions include water, and alcohols, such as propylene glycol, glycerol, sorbitol and their mixtures. Examples of emulsifiers that may be used in preparing the emsulsions include include non-ionic surfactants, for example polyoxyethylated castor oil, polyoxyethylated sorbitan monooleate, sorbitan monostearate, glycerol monostearate, polyoxyethyl stearate, alkylphenol polyglycol ethers; ampholytic surfactants such as di-sodium N-lauryl-.beta.-iminodipropionate or lecithin; anionic surfactants such as Na lauryl sulphate, fatty alcohol ether sulphates, the monoethanolamine salt of mono/dialkyl polyglycol ether orthophosphoric esters; and cationic surfactants such as cetyltrimethylammonium chloride.

The pharmaceutical compositions of the present invention may include a therapeutically effective amount of one, or more than one steroidal anti-inflammatory drug, such as hydrocortisone, and/or one, or more than one non-steroidal anti-inflammatory drug. In addition, the pharmaceutical compositions of the present application may include an effective amount of a topical anesthetic for numbing of the skin of the animal. Non-limiting examples of topical anesthetics that can be used in the compositions of the present invention include benzocaine, butamben, dibucaine, lidocaine, oxybuprocaine, pramoxine, proparacaine, proxymetacaine, and tetracaine.

The pharmaceutical compositions of the present invention may also contain a therapeutically effective amount of a cereal β glucan, which can help seal, protect and moisturize the skin of the animal, stimulate fibroblast growth and help promote the healing and repair of the skin.

The amount of cereal β glucan in the pharmaceutical compositions of the present invention may be from about 0.001 wt % to about 1 wt % or any value or subrange therebetween, from about 0.01 wt % to about 0.8 wt % or any value or subrange therebetween, or from about 0.1 wt % to about 0.5 wt % or any value or subrange therebetween.

The pharmaceutical compositions of the present invention may be prepared using cereal β glucan solutions containing from about 0.01 wt. % to about 1.2% wt. %, from about 0.1 wt. % to about 1.1 wt. %, or from about 0.5 wt. % to about 1 wt. % of the cereal beta glucan. These beta glucan may be prepared from a beta glucan having a purity of from about 65% to about 100%, from about 75% to about 100%, or from about 85% to about 100%, which contains less than 20%, less than 15%, less than 10%, or less than 5% of impurities, such as protein, lipid, carbohydrate, and particulate impurities.

In further examples, the one, or more than one avenanthramide may be present in the pharmaceutical compositions of the present invention at a concentration of from about 0.0001 to about 375 ppm or any value or subrange therebetween, 0.001 to about 375 ppm or any value or subrange therebetween, from about 0.0001 to about 150 ppm or any value or subrange therebetween, from about 0.001 to about 150 ppm or any value or subrange therebetween, from about 0.01 to about 150 ppm or any value or subrange therebetween, from about 0.01 to about 50 ppm or any value or subrange therebetween, from about 0.3 to about 15 ppm or any value or subrange therebetween, or from about 1.5 to about 4.5 ppm or any value or subrange therebetween.

In another example, the pharmaceutical compositions of the present invention may comprise from about 0.1 to about 25 weight percent or any value or subrange therebetween, or from about 1 to about 10 weight percent or any value or subrange therebetween, of an oat extract comprising the one, or more than one avenathramide at a concentration of from about 1 to about 1500 ppm or any value or subrange therebetween, or from about 3 to about 450 ppm or any value or subrange therebetween, based on the oat extract.

The amount of the ecto- and/or endo-parasiticidal agents present in the therapeutic/parasticidal compositions of the present invention may be from about 0.001 wt % to about 20 wt % or any value or subrange therebetween, from about 2 wt % to about 15 wt % or any value or subrange therebetween, or from about 5 wt % to about 10 wt % or any value or subrange therebetween.

Example 12 demonstrates that β(1-3) β(1-4) glucan prepared according to the method described in U.S. patent application Ser. Nos. 10/554,288 and 10/554,290, the disclosures of which are incorporated herein by reference, and applied in the form of a topical composition to the surface of a section of skin, can significantly cross into the horny layer, the epidermis, the dermis and the subcutis layers of the skin. These results suggest that a parasiticidal agent encapsulated by the β(1-3) β(1-4) glucan isolated according to this method could also be effectively transferred down to the dermis and subcutis layers of the skin of a subject. As a result, the therapeutic/parasiticidal compositions of the present invention, which include a β(1-3) β(1-4) glucan, are advantageous in that they can readily transport ecto- and/or endo-parasiticidal agents to the dermis and subcutis layers of the skin of an animal.

The pharmaceutical compositions of the present invention may also contain various known and conventional therapeutic and/or cosmetic ingredients providing they do not detrimentally affect the desired reduction of skin irritation. For example, cosmetic ingredients such as alcohols, fats and oils, surfactants, fatty acids, silicones, humectants, moisturisers, viscosity modifiers, emulsifiers, stabilisers, colourings agents, and perfumes or fragrances may be included.

Cereal β glucans suitable for use in preparing the compositions of the present invention are available in powdered form from several commercial suppliers, such as Sigma Chemical Co. (St. Louis, Mo.) and Ceapro Inc. (Edmonton, AB, Canada). Solutions of beta glucan can be prepared in the manner described in U.S. Pat. No. 6,284,886 or in U.S. Patent Application Publication No. 2006/0122149.

The cereal beta glucan content of the compositions of the present application can be determined using a number of methods, known to those skilled in the art. For example, beta glucan content can be assessed colorimetrically and/or by standard analytical techniques such as size exclusion chromatography and HPLC (see Wood et al. Cereal Chem. (1977) 54:524; Wood et al. Cereal Chem. (1991) 68:31-39; and Wood et al. Cereal Chem. (1991) 68:530-536). Beta glucans can also be analyzed enzymatically using commercially available kits, such as Megazyme (freland) employing the techniques of McCleary and Glennie-Holmes J. Inst. Brew. (1985) 91:285.

Viscosities can be measured with a rotational, shear-type viscometer such as the Brookfield Syncro-Lectric or the Haake Rotovisco. Methods of using the instrument are known to those skilled in the art. Routinely, measurements are made at four speeds of disc rotation at a constant temperature of 25° C.

The oat extract, which may be used to prepare the therapeutic or therapeutic/parasiticidal compositions of the present invention, may be produced according to the method of the present invention. Thus, according to a further aspect of the present invention, there is disclosed a method for producing of an oat extract comprising the following steps:

a. Milling whole oats,

b. Extracting the resulting oatmeal or milled oat fraction with a solvent,

c. Adjusting the pH of the resulting oat extract to <4.0 (favorably <3.5),

d. Membrane filtration (e.g. ultra-filtration) of the oat extract through a membrane <10⁴ MWCO.

In another aspect of the present invention, there is provided an oat extract containing a minimum of 10 ppm of avenathramide, wherein the oat extract can be produced by a method comprising steps a-d as above, and the additional step

e. Adjusting the concentration of avenanthramide in the permeate after membrane Filtration to >10 ppm.

In accordance with the present invention, an intermediate oat extract can be prepared by milling whole oats, extracting the oatmeal by mixing with a solvent, separating the resulting intermediate extract from the spent grain and adjusting the pH of the intermediate extract to <4.0 (preferably <3.5). The pH adjustment leads to high avenanthramide yields in the extract while providing good stability to the extract

Once extracted and acidified the intermediate oat extract is stable for greater than 12 months.

The intermediate extract is subjected to membrane filtration, preferably ultra-filtration, whereby the filtrate of <10,000, more preferably <5,000 molecular weight is collected. The final resulting oat extract may be further concentrated by, for example, reverse osmosis (RO) to increase the avenanthramide concentration to, for example, >0.1% (d.w.b.).

The final resulting oat extract may be used for therapeutic or cosmetic purposes directly in alcohol. Alternatively it may be subjected to solvent exchange and the extract made up in a solvent of choice including, but not limited to, for example, butylene glycol, pentylene glycol, propylene glycol, glycerol, mixtures of these solvents, and combinations of these solvents or solvent mixtures with water.

The final resulting oat extract is readily formulated as a solution, gel, lotion, cream, ointment, powder or other pharmaceutically acceptable form. Preparations are formulated using methods known to those skilled in the art. For a reduction of erythema, the compositions should contain about 1-3% of the liquid oat extract (provided as a standardized 15 ppm avenanthramide solution).

Primarily, the present invention provides a method for the production of an oat extract that offers several advantages over the known methods of extraction and enhances the properties of the extract.

Histological staining of intact oat kernels indicated that the phenolic compounds were located primarily in the aleurone layer of the oat kernel. This implied that enriched preparations of the functional compounds would best be made from bran obtained by conventional milling or debranning processes. It was surprisingly determined that the maximum yield of avenanthramides came from the whole oat, not a bran fraction.

The present invention is based on the discoveries that (a) the extraction of active ingredients from oat may be enhanced in terms of production and efficiency, and furthermore (b) the resulting extracts are stable for extended shelf-life periods and may be concentrated readily.

Accordingly, the present invention provides a process for producing an oat extract, comprising:

a. Milling whole oats,

b. Extracting the resulting oatmeal or milled oat fraction with a solvent,

c. Adjusting the pH of the resulting oat extract to <4.0 (favorably <3.5), d. Membrane filtration of the oat extract with a pH <4.0 through a membrane <10⁴ MWCO.

The oat extract produced according to the method of the present invention is quantifiable in terms of activity and certified product quality assurance can be given. In accordance with the invention, aqueous alcoholic extracts of whole oats or groats are refined to provide materials for use in cosmetic and pharmaceutical compositions such as creams, gels, powders, lotions, and the like.

The oat extract of the present invention can contain avenanthramides at a concentration of between 1 and 1500 ppm of avenanthramide, between 3 and 450 ppm of avenanthramide, and or between 15 and 150 ppm of avenanthramide. Other compounds, for example phenolics, benzoic and cinnamic acids, flavones, flavonols, chalcones, flavanones, proanthocyanidins, aminoplienolics, tocols, and saponins, may also be found in the oat extract. These compounds may have utility as for example, antioxidants, antimicrobials, antifungals, sunscreens, and surfactants.

The oat extract according to the present invention contains no or very little amounts of β glucan, for example less than about 0.01%, and less than 0.01% protein of molecular weight greater than 10,000 Da. Residual concentrations of protein and starch in the oat extract are dependent on the concentration of avenanthramides in the extract.

In step d of the method of preparing avenanthramides according to the present invention, the membrane filtration is an ultra-filtration. In addition, reverse osmosis may be used to further concentrate and purify the oat extract obtained by step d.

In step b, the solvent for extracting the oatmeal may comprise water and a alcohol. The alcohol may be selected from the group consisting of ethanol, methanol, propanol (n-, iso-), butanol (n-, iso-, tert-), or a mixture thereof, such as ethanol:water.

The oat extract may be incorporated into a solvent for ease of handling. For example, the oat extract may be incorporated in a 1:1 w/w mixture of 1,3 butylene glycol, propylene glycol or glycerol and water.

The oat extract obtained according to the method of the present invention can be easily sterilised by heat, microfiltration, or irradiation (after step c or d).

The following examples are provided to exemplify the present invention. Variations and alterations will be readily apparent to those skilled in the art.

### Examples

### Example 1

### Oat Extract Preparation Process

Two or three replicates for each method were processed and analysed.

METHOD. Oat groats (Variety Hinoat) were ground through a Willey Mill to pass through a 10 Mesh screen. Oatmeal at a mixing ratio of 1:4 (w/v) oatmeal:solvent was added to a stirred solution of 50% (v/v) aqueous ethanol at 40 C. The resulting mixture was stirred for 30 minutes and then cooled to room temperature. The mixture was then centrifuged at 2830 g for seven minutes and the supernatant drawn off. The pellet was re-suspended in fresh solvent and re-centrifuged. The supernatant was drawn off and the pellet re-suspended a third time in fresh solvent. All supernatants were combined and filtered through a course sintered glass filter.

To show the difference between the method (process) for producing an oat extract according to the present invention, which comprises the step of adjusting the pH of the extract to <4.0, and a method which does without pH adjustment, a comparison test series was carried out. Test samples were designated UF-B1, UF-B3, UF-C1, UF-C2, and UF-C3, respectively.

For samples of the I.D. series UF-B1 (comparison samples), in contrast to the method according to the present invention the oat extract was applied directly to the ultrafiltration module.

For samples of the series UF-B3, UF-C1, UF-C2, and UF-C3, in accord with the present invention the pH of the extract was adjusted to 2.5 with hydrochloric acid (IN) and ethanol added ˜1%) to clarify the solution. The pale yellow extract was passed through a 0.45 μm filter (Gelman; Supor DCF) before ultrafiltration.

For ultrafiltration a Millipore Corporation MINI-PLATE™ Tangential-Flow Bioconcentrator (10,000 MWCO) was used. The unit contains a low protein binding YM membrane with a surface area of 108 cm². Pump rate was 1000 ml/min. and the flux (flow) was typically 14 L/m²/h (LMH).

Weight profiles were conducted on the sample ID series UF-B by lyophilisation for 72 hours.

ANALYSIS High Performance Liquid Chromatography (HPLC) analysis was performed using a Thermo Separations Products (TSP) Spectra P4000 pump, a Varian column oven, and a Waters 991 Photodiode Array (PDA) detector with accompanying software. The column used was a CSC-Hypersil (51 .mu.m, 120 A, 0.46×25 cm-serial #039775) at 25° C. UV monitoring at 330 nm was used. The flow rate was set at 1.0 ml/min.

All samples and standards were prepared in ethanol/water (1:1).

| | |
|---|---|
| AF-1 standard (0.1 μg/μl): 5 μl injected | Retention time: 23.68 minutes |
| AF-2 standard (0.1 μg/μl): 5 μl injected | Retention time: 26.95 minutes |

avenanthramide fractions were prepared in 50% ethanol/water (5 ml) and 5 μl injected

Table 1 describes the HPLC solvent program for the analysis of avenanthramides.

TABLE 1

| | | | 5% Acetic |
|---|---|---|---|
| Time (min.) | MeOH | H₂O | Acid |
| 0 | 40 | 55 | 5 |
| 40 | 55 | 40 | 5 |
| 45 | 85 | 10 | 5 |
| 50 | 100 | 0 | 0 |
| 53 | 40 | 55 | 5 |
| 55 | 40 | 55 | 5 |

Results As provided in Table 2 total avenanthramides were calculated and expressed as AF-I equivalents and recovery efficiency expressed as percentage recovery of avenanthramides from the permate are based on total avenanthramides.

TABLE 2

| | UF Method | | | | | | |
|---|---|---|---|---|---|---|---|
| Sample | | | | Clean | | | Conc. |
| I.D. | pH | Permeate | Retentate | in place | Recovery | Diafiltration | polarization |
| UF-B1P | 7.5 | 33.8 | — | — | 57% | No | No |
| UF-B1R | 7.5 | — | 21.8 | — | 37% | No | No |
| UF-B3P | 2.5 | 45.5 | — | — | 77% | No | No |
| UF-B3R | 2.5 | — | 8.1 | — | 14% | No | No |
| UF-B3C | 2.5 | — | — | 1.1 | 2% | No | No |
| UF-C1P | 2.5 | 38.6 | — | — | 75-109% | Yes | No |
| UF-C2P | 2.5 | 43.3 | — | — | 84-122% | Yes | Yes |
| UF-C3P | 2.5 | 42.5 | — | — | 82-120% | Yes | Yes |

Notes:
1. Values based on AF-1 equivalents
2. Percent Avenanthramide recoveries of the permeate fraction for the C-Series are given as a range from UF-C1, C2, and C3 values

Qualities of the Oat Extract

1. No haze formation has been observed in any oat permeate extracts produced to date.

2. Efficiency of the avenanthramide extraction is >75%, more typically 85-100%.

3. The oat extract can be concentrated up to 50-fold without precipitation occurring.

4. The oat extract has low or no bacterial counts due to the permeate feed-stream being sterile before concentrating.

5. The transparent oat permeate extract has a pale yellow colour with a shelf life of more than 12 months.

6. The oat extract has a pleasant oat odour.

7. The permeate fraction was readily soluble at neutral pH in 35-70% ethanol/water.

### Example 2

### Oat Extract Process Scale-Up.

METHOD Oat groats (Variety AC Ernie) were ground through a Willey Mill to pass through a 10 Mesh screen seive. Oatmeal (1.5 kg) was added to a stirred solution of 50% (v/v) aqueous ethanol (6000 ml) at 40° C. The resulting mixture was stirred for 30 minutes and then cooled to room temperature. The mixture was then centrifuged at 2830 g for seven minutes and the supernatant drawn off. The pellet was re-suspended in fresh solvent (3000 ml) and re-centrifuged. The supernatant was drawn-off and the pellet re-suspended a third time in fresh solvent (3000 ml). All supernatants were combined and filtered through a coarse sintered glass filter. The pH of the extract was adjusted to pH 3.5 with hydrochloric acid (1M) and ethanol added (−1%) to clarify the solution. The pale yellow extract was passed through a 0.45 μm filter (Gelman; Supor DCF) and made up to 12000 ml before ultrafiltration.

The extract was ultrafiltered at ambient temperature through a modified PES (Omega) T-screen membrane (0.09 m²; 5000 MWCO, Pall Filtron) using a Pall Corporation CENTRASETTE™ unit. Flux rates (flow rates) ranged from 20-25 LMH. The pH of tile resulting permeate was adjusted back to 6.5 with aqueous potassium hydroxide (SM).

A 200 ml aliquot was evaporated to dryness under reduced pressure and made up to 10 ml in 1:1 (v/v) aqueous ethanol. The solution was applied to a calibrated open column containing 100 mls. of LH-20 chromatographic gel (AP Biotech, Sweden) pre-equilibrated in ethanol:water:acetic acid (40:59:1). The column was washed with 2 Vb of solvent and the resulting fraction discarded. The avenanthramides were eluted from the column with 2 bed volumes of 80% aqueous acetone. The sample was evaporated to dryness under reduced pressure and made up in 1:1 aqueous ethanol (5 ml). The sample was filtered through a 0.45 μm filter into a screw-capped vial for HPLC analysis.

ANALYSIS HPLC analysis for total avenanthramides was conducted using a Thermo Separations Products (TSP) solvent delivery system and Hewlett Packard (HP) data collecting software on a C 18 CSC HYPERSIL™ column (250×4.6 mm, 120 A, 3 μm). An HP photodiode array (PDA) detector monitoring from 190400 nm, and specifically at 340 nm was used to detect all avenanthramides. All peaks were integrated using retention times relative to an authentic AF-1 standard (obtained from Agriculture and Agri-Food Canada, ECORC, Ottawa, Canada). The solvent system consisted of acetonitrile, water, and aqueous 5% acetic acid as shown in Table 3.

TABLE 3

| | | | 5% Acetic |
|---|---|---|---|
| Time (min.) | Acetonitrile | H2O | Acid |
| 0-20 | 25 | 70 | 5 |
| 20-25 | 100 | 0 | 0 |
| 25-30 | 25 | 70 | 5 |
| 30-35 | 25 | 70 | 5 |

To complete product formulation 3382 ml of permeate feedstream was concentrated to dryness under reduced pressure and made up to 2000 ml (90% aqueous 1,3 butylene glycol) and 0.3% (w/w) phenoxyethanol added. The solution was filtered through a 0.45 μm filter (Whatman) before packaging. The finished oat extract contains 10 ppm of total avenanthramides.

### Example 3

### Anti-Erythema Testing in Human Subjects

Skin tests were carried out on healthy male and female volunteers

a. 18 to 60 years of age;

b. Fair-skinned with skin types I-III, determined by the following guidelines:

I Always burns easily; never tans (sensitive)

II Always burns easily; tans minimally (sensitive)

III Burns moderately; tans gradually (normal)

IV Burns minimally; always tans well (normal)

V Rarely burns; tans profusely (insensitive)

VI Never burns; deeply pigmented (insensitive)

The following exclusion criteria were followed:

a. Subjects with a history of abnormal response to sunlight;

b. Subjects exhibiting current sunburn, suntan, or even skin tone which might be confused with a reaction from the test material or which might interfere with evaluation of the results of the test;

c. Pregnant or lactating females;

d. Subjects taking medication which might produce an abnormal response to sunlight or interfere with the results of the test;

e. Subjects who regularly use UVA sunbeds; or

f. Subjects exhibiting any visible skin disease which could be considered to affect the purpose or integrity of the study.

Nine (9) subjects who met the inclusion criteria were selected for participation.

A xenon arc solar simulator (Solar Light Source, Philadelphia, Pa.) was used as the source of ultra-violet light. A continuous emission spectrum in the UV range (290-400 nanometres) was utilised during the course of this testing procedure. The lamp output was measured with a UV intensity meter (Model PMA 2100) with the appropriate detector attached.

A Minolta CHROMA METER™ CR-300 (Minolta Corporation Ltd., Osaka, Japan) was used to measure erythema levels. The a* value of the L*a*b* colour notation system is indicative of colour changes in the red-green colour axis. The higher the value, the more intensely red the object being evaluated. Therefore, the a* value was used as a measure of redness (erythema) on the skin surface. An increase in a* values is considered indicative of increased erythema.

On day 1 the minimal erythemal dose (MED) of each subject was determined by a progressive sequence of timed UV light exposures, each of which was graduated incrementally by 25% over that of the previous site. An MED is defined as the time interval or dosage of UV light irradiation sufficient to produce a minimal, perceptible erythema on untreated skin.

On day 2 subjects returned to the laboratory approximately 24 hours after irradiation for determination of their MEDs. The sites were evaluated for erythema according to the following visual scoring criteria:

0=negative, no visible reaction

0.5=minimal erythema

1.0=defined erythema

2.0=moderate erythema

3.0=severe erythema

A technician outlined seven 1″.times.1.5″ test-sites areas on each subject's back, between the scapulae and the belt-line, lateral to the mid-line, with a surgical marking pen. Six test sites were designated for the test materials and one for the untreated irradiated control.

The sites were then exposed to UV light 1.5 times the pre-determined MED values.

On day 3, approximately 24 hours after irradiation, erythema was evaluated and scored visually by a trained technician using the criteria outlined above. Baseline a* value readings were also taken with the Minolta CHROMA METER™. Three consecutive chroma meter readings were taken and averaged.

Approximately 0.2 ml of test product was applied to the appropriate test site. Approximately 4 hours after product application, the test sites were visually scored and Minolta chroma meter reading taken.

On day 4 tile subjects returned to the clinic approximately 24 hours after the product application. The 7 sites were again evaluated for erythema using both the visual grading system and the Minolta CHROMAMETER™

The results were subjected to statistical analysis using t-Test (dependent) to determine if any significant differences were observed in the mean chroma meter a* value readings from baseline (24-hours post-irradiation) to 4-hours post-treatment and 24-hours post-treatment, for each test site. Significance was observed if p≦0.05.

Product test solutions consisted of oat extract in butylene glycol:water 1:1 w/w adjusted to the required concentration (ppm) of avenanthramide.

The results of testing oat extract in human volunteers are shown in Table 4.

TABLE 4

| | | | | | Change from | |
|---|---|---|---|---|---|---|
| Oat Extract | Average a* Value | | | | Baseline (%) | |
| Avenanthramide | | Base- | 4 | 24 | 4 | 24 |
| (PPM) | | line | Hours | Hours | Hours | Hours |
| 45.0 | Site #2 | 11.47 | *10.39 | *9.33 | −9.4 | −18.7 |
| 15.0 | Site #3 | 12.47 | *11.03 | *10.18 | −11.5 | −18.4 |
| 5.0 | Site #4 | 12.65 | 11.30 | *10.19 | −10.7 | −19.4 |
| 1.5 | Site #5 | 12.04 | *10.67 | *10.35 | −11.4 | −14.0 |
| 0.5 | Site #6 | 12.42 | *11.10 | 11.54 | −10.6 | −7.1 |
| Untreated | Site #7 | 13.22 | *12.03 | 12.53 | −9.0 | −5.2 |
| Irradiated Control | | | | | | |

Note
*denotes statistically significant difference from baseline readings

The tests indicated that the oat extracts were efficient at reducing erythema. The dose response kinetics indicated that between 0.03 and 0.3 ppm the relationship between dose and response was linear. Maximum response was obtained at >0.3 ppm of Avenathramide.

### Example 4

### Isolation and Purification of an Avenanthramide Fraction

Further to Example 2, the permeate (270 ml) was evaporated under reduced pressure and made-up to 10 mls in 1:1 (v/v) aqueous ethanol. The solution was applied to a LH-20 column (100 ml) pre-equilibrated in ethanol:water:acetic acid (40:59:1). The column was washed with 2 Vb of solvent and the resulting fraction discarded. The avenanthramides were eluted from the column with two bed volumes of 80% aqueous acetone. The sample was evaporated to dryness under reduced pressure and then redissolved in 100 mls of 90% aqueous butylene glycol. The solution was filtered through a 0.45 .mu.m filter (Whatman Inc.) before packaging. The finished, isolated avenanthramide fraction contained 15 ppm of total Avenathramide.

The results of testing the isolated avenanthramide fraction, oat extract, and untreated control are shown in Table 5.

TABLE 5

| | | | | | Change from | |
|---|---|---|---|---|---|---|
| | Average a* Value | | | | Baseline (%) | |
| | | Base- | 4 | 24 | 4 | 24 |
| Sample | | line | Hours | Hours | Hours | Hours |
| Isolated | Site #1 | 12.62 | 11.95 | *10.74 | −5.3 | −14.9 |
| Avenanthramide | | | | | | |
| (15.0 ppm | | | | | | |
| Avenanthramide) | | | | | | |
| Oat Extract | Site #3 | 12.47 | *11.03 | *10.18 | −11.5 | −18.4 |
| (15.0 ppm | | | | | | |
| Avenanthramide) | | | | | | |
| Untreated | Site #7 | 13.22 | *12.03 | 12.53 | −9.0 | −5.2 |
| Irradiated Control | | | | | | |

Note:
*denotes statistically significant difference from baseline readings

### Example 5

### Rapid Analytical Method for Avenathramide

High Performance Liquid Chromatography (HPLC) for total avenanthramides was conducted using a Beckman binary solvent delivery system using 32 KARAT™ analytical software for Microsoft WINDOWS NM™ (Beckman Coulter Inc.). avenanthramides were separated on a CSC ODS HYPERSIL™ column (250×4.6 mm, 120 Å, 3 μm ) using a C 18 guard column (Supelco: Sigma-Aldrich Corporation) at 22 C. A Beckman photodiode array (PDA) detector monitoring from 210-400 nm, and specifically 330 nm was used to detect all avenanthramides. The peaks of three major avenanthramides; AF-1, AF-2, and AF-6 were integrated using retention times and spectral data relative to authentic standards synthesized by Symrise AG.

Extracts were diluted in equal portions with distilled water and stored at 4 C in amber sample vials before analysis. Twenty (20 .mu.l aliquots) were injected in triplicate. The HPLC solvent system consisted of acetonitrile, and 0.01 M aqueous phosphoric acid is shown in Table 6.

TABLE 6

| Time (min.) | Acetonitrile (%) | 0.01 M Phosphoric Acid |
|---|---|---|
| 0 | 25 | 75 |
| 20 | 37 | 63 |
| 22 | 100 | 0 |
| 25 | 100 | 0 |
| 28 | 25 | 75 |
| 33 | 25 | 75 |

### Example 6

### Large Scale (Commercial) Production of Oat Extract

Method. Hulless oats, 500 kgs (variety N0141-1) frozen overnight at −18 C. The frozen grain was ground through a FITZ MILL® COMMINUTOR® (The Fitzpatrick Company: Elmhurst, Ill.) equipped with a ⅛^{th} inch screen to produce a coarse oatmeal (100% passed through a 10 Mesh and <10% passed through a 100 Mesh screen sieve).

The meal was vigorously dispersed in 1500 kg of 50% (w/w) ethanol at 20° C. and mixed for 0.5-16 hours. The resulting slurry was centrifuged through a decanter centrifuge (Westphalia Separator). The pH of the supernatant was adjusted to pH 2.5-4.5 with hydrochloric acid (17.5% w/w) and stirred for 30-60 minutes.

The extract was then subjected to ultrafiltration using 5,000 MWCO spiral membrane (21.4 m² Synder Filtration, Vacaville, Calif.).

The sterile permeate was next concentrated using reverse osmosis (RO) membrane filtration (15 m² FilmTec Corporation, Minneapolis, Minn.). Before RO concentration the pH was adjusted to ph 6±0.5). Following concentration the resulting oat extract had an avenanthramide concentration of between 200 and 1500 ppm. This extract was found to be stable for more than fourth months with no loss of activity, clarity or other measurable parameters of product quality.

The high avenanthramide extract was used as a stock solution for direct use in therapeutic or cosmetic formulations, or alternatively, the ethanol:water was replaced with an alternative solvent for example butylene glycol:water or glycerol:water.

### Example 7

Formulation of Oat Extract Concentrate into Butylene Glycol:Water

A diluent solution was prepared by taking >90% of the required final volume of butylene glycol:water (50% w/w) to which is added the calculated volume of oat extract concentrate. The required volume of concentrate is readily calculated from the values of concentrate Avenathramide concentration, together with the final desired concentration and volume. Oat extract has been formulated into butylene glycol:water at Avenathramide concentrations in the range of 15-200 ppm of avenanthramide.

The product was thoroughly mixed and then heated to 70 C. The product was then passed through an evaporator (Pfaudler, Inc. Wiped Film Evaporator) to remove ethanol. Residual ethanol was tested for using standard gas chromatographic (GC) techniques. Following passage through the evaporator, the butylene glycol:water ratio was checked and adjustments made to account for any loss of water in the evaporator. For cosmetic and therapeutic use the value of pH of the product was adjusted to 6.0-7.5.

Finally, the preservative 2-phenoxyethanol was added (0.3% w/w) to the product. The product was sterilized by membrane filtration. The product avenanthramide content was then analysed and confirmed to meet the desired product specification.

### Example 8

Formulation of Oat Extract Concentrate in Glycerol:Water

A diluent solution was prepared by taking >90% of the required final volume of glycerol:water (>30% w/w) to which is added the calculated volume of oat extract concentrate. The required volume of concentrate is readily calculated from the values of concentrate Avenathramide concentration, together with the final desired concentration and volume. Oat extract has been formulated into glycerol:water at Avenathramide concentrations in the range of 15-250 ppm of avenanthramide.

The product was thoroughly mixed and then heated to 70 C. The product was then passed through an evaporator (Pfaudler Wiped Film Evaporator) to remove ethanol. Residual ethanol was tested for using standard gas chromatographic techniques. Following passage through the evaporator, the glycerol:water ratio was checked and adjustments made to account for any loss of water in the evaporator. For cosmetic and therapeutic use the pH of the product was adjusted to pH 6.0-7.5. For functional food/nutraceutical use the pH of the product was adjusted to pH 4.0.

Finally, the preservative system consisting potassium sorbate (0.1% w/w) and sodium benzoate (0.1% w/w) was added to the product. The product avenanthramide content was then analysed and confirmed to meet the desired product specification.

### Example 9

### Hypo-Allergenic Shampoo for Use

Table 7 presents an example of a therapeutic shampoo formula falling within the scope of the present invention with amounts provided expressed as weight percent.

TABLE 7

| | | | Percent by |
|---|---|---|---|
| Phase | Material Description | Supplier | weight |
| A | Deionised water | | 45.65 |
| A | Sequestrene NA3T | Ciba-Geigy | 0.05 |
| A | Incromide LR | Croda Inc. | 5.00 |
| A | Standapol ES-2 | Henkel | 28.00 |
| A | Velvetex BA-35 | Henkel | 8.00 |
| A | Polysorbate 20 | ICI | 1.50 |
| B | Hydrolysed Oat Protein | Ceapro Inc. | 8.00 |
| B | Oat Extract | Ceapro lnc./DRAGOCO | 3.20 |
| | | Gerberding & Co. AG | |
| B | Oat Beta Glucan | Ceapro Inc./DRAGOCO | 0.20 |
| | | Gerberding & Co. AG | |
| C | Fragrance | | 0.20 |
| C | Kathon CG | Rohn and Haas | 0.20 |

Add ingredients in phase A one at a time with medium agitation at room temperature. Ensure each ingredient is dissolved before adding next. The solution should be clear before going onto phase B. In phase B, add ingredients one at a time to phase A with mixing. Add ingredients in phase C one at a time to the mixing phase AB. Adjust the pH with a 50% solution of citric acid until the pH is 6.5.

To use, the product may be either applied directly to the animal or alternatively, mixed with water in a suitable vessel and applied to the animal by sponging. The product rinses easily ensuring that all surfactant is removed after bathing.

The completed shampoo effectively reduced pruritus in mammals. Further, the shampoo reduced shedding and scaling.

### Example 10

### Soothing Formula for Use in Treating Otitis

Table 8 presents an example of a pharmaceutical cleansing formula falling within the scope of the present invention with amounts provided expressed as weight percent.

TABLE 8

| Ingredient | % Formula |
|---|---|
| Deionised water | 46.0 |
| Butylene glycol | 48.85 |
| Oat Extract | 4.0 |
| Lactic Acid | 0.8 |
| Malic Acid | 0.2 |
| Methyl Paraben | 0.15 |

The ingredients were added one at a time to a mixing vessel with stirring. Ensure each ingredient is dissolved before adding next. The pH of the finished product was adjusted to 4.0 using 50% malic acid.

The product is for use in cleaning ears in dogs, puppies, cats, and kittens.

To clean the ear, fill the canal with cleanser, flip the ear pinna over, and massage. Take cotton balls and thoroughly remove exudate and dry the accessible portion of the canal. Repeat daily until ear is clean, treat weekly afterwards or as directed by the veterinarian.

Clinical trial results proved the product to be superior in reducing redness associated with otitis and to effectively reduce irritation, promoting the healing of the mammal.

### Example 11

### Method for Purifying Cereal β-Glucan Derived from Oat Bran

Oat bran (The Quaker Oats Company) was slurried with alkaline reverse osmosis (RO) water at a pH of about 9.5 to a final solids concentration of 4-10%. The temperature was maintained at 45° C.±5° C. The cereal β-glucan was extracted from the oat bran over a period of 30 minutes. After this time, the solids were removed by centrifugation with a decanter centrifuge. The centrate was cooled to room temperature, and the cationic flocculant SURFLOC® 34030 (Jes-Chem Ltd.) was added at a 0.2% concentration. Following an incubation period of 20 minutes, coagulated particulate material was removed by centrifugation using a disk-stack centrifuge. The pH of the centrate was adjusted to approximately neutral, heated to >72° C. to gelatinize starch, and treated with TERMAMYL® LC (Novozymes A/S), a heat-stable α-amylase enzyme for starch liquefaction at low calcium levels. When the solution no longer produced a positive iodine test, the pH was reduced to about 4.0 to inactivate the enzyme, and the mixture was heated to 85° C. for 30 minutes to denature the protein present. The solution was cooled to 4° C. for one hour, and then heated to a temperature of about 72° C. An equivalent weight of CELPURE® C300 (diatomaceous earth having a permeability of 0.300 Darcy; World Minerals) was added to the solution, and the mixture was then filtered using a filter-press containing 25 μm filter-papers and pre-coated to a depth of about 4 mm with CELPURE® C65 (diatomaceous earth having a permeability of 0.065 Darcy; World Minerals). The filter press was preheated to a temperature of about 65° C., and the pH of the feedstream for the filter press was adjusted to 4.5 before the β-glucan solution was filtered. After the β-glucan solution was passed through the filter, the press was flushed with reverse osmosis water resulting in a clear, pale yellow coloured β-glucan solution. The β-glucan solution was cooled to 5° C. and 95% ethanol at a temperature of −20° C. was added to a final volume of about 15% (w/w) with stirring. A suspension of β-glucan was formed that was immediately separated from the solution by centrifugation with a disk-stack centrifuge. The isolated solid β-glucan was added to RO water at 45° C., allowed to disperse and then heated to between 60-70° C. to produce a clear colorless solution containing about 1% β-glucan. The separated β-glucan was colourless, had a purity of greater than 75%, a viscosity >500 cP, and an exception clarity <50 NTU, as measured using a turbidity meter.

### Example 12

### Quantification of the Distribution of Purified β-Glucan Applied as an Aqueous Composition to Abdominal Skin Sections

Human abdominal skin was received under informed consent from five healthy donors having undergone plastic surgery. The skin from each patient was liberated from subcutaneous fat, and cut into three sections. The skin sections were frozen in liquid nitrogen and sterilized overnight with a dose of 25 kGy of gamma-radiation. The irradiated samples were each mounted in a 20 mL volume FRANZ-CELL®-like perfusion chamber (PHACOCELL®, PhaCos GmbH, D-82131-Gauting, Germany; see Artmann, C. W. In vitro percutaneous absorption into human skin, Fundam. Appl. Toxicol., 28, 1-5 (1996)) containing an acceptor medium. Using a microdose applicator, the irradiated samples of skin were coated with a 5 mg/cm² dosage of Composition 1455, Composition 1450 or a control composition. The Compositions 1455 and 1450 were aqueous compositions containing 5% and 50%, respectively, of the β(1-3) β(1-4) glucan prepared according to the isolation method of the present invention (see Example 11). The control composition was an aqueous composition that did not contain any β(1-3) β(1-4) glucan. The chamber was kept free of air bubbles while filling in order to ensure complete and even rinsing of the skin tissue. Pressure compensation, inside and outside of the chamber and a constant humidity of air was provided by ventilation. The skin temperature was monitored with temperature sensors, and the moisture content of the skin sections was monitored with a corneometer. The medium was regulated at 36° C. and circulated continuously. Skin humidity was kept at about 65 corneometer units, and the skin surface temperature was kept at 32° C. via a ventilation channel. The above conditions were maintained by regulation of the temperature of the medium by using a heating plate at the base of the chamber, and air tubes, and by adjusting the flow of air in the chamber. The skin sections were supplied by the uniformly circulating nutrient medium, which rinsed their lower surfaces. The area of application for all samples was fixed at 10 cm². The skin samples were incubated for eight hours under non-occlusive (open) conditions.

At the end of the incubation period, swab samples of the skin sections were taken with both dry cotton gauze swabs and cotton gauze swabs moistened with 0.2 mL of 70% methanol/H₂O. The skin sections were removed from the PHACOCELL® chamber and immediately frozen in liquid nitrogen. The skin sections were then cut into 15 μm slices from the horny layer to the deeper dermis. The skin sections were allowed to air dry on clean glass slides and not fixed with any fluid. The slices were then stained with BACTIDROP™ Calcofluor White for 30 seconds and then washed of excess stain with deionized water. The staining and washing steps were repeated twice. The stained sample was covered with a clean glass cover slip and examined by fluorescence with a LEIKA® fluorescent microscope having an exciter filter ranging between 400-500 nm with a peak of 440 nm, a barrier filter of 500-520 nm, and a xenon arc (burner) lamp. BACTIDROP™ Calcofluor White is a non-specific fluorochrome that binds to cellulose, and upon excitation with long wavelength ultraviolet light delineates the cell walls of cellulose-containing organisms. The deposition of the β-glucan molecules was monitored and quantified using bright fluorescence, focus inverted to white spots (3-5 μm) seen upon the cell walls of the samples and in the intercellular interstices.

The mean percent depositions as determined by the above fluorescence staining method are shown in Table 9. Significant fluorescent staining values (>5%) were observed in the horny layer and in the epidermis of the skin samples treated with Composition 1455 and Composition 1450. Relatively lower values were observed in the dermis and subcutis layers of the skin samples treated with Composition 1450 and Composition 1455. Fluorescence staining values of <1% were observed with the skin sections that were treated with the control composition.

TABLE 9
Mean Percent Deposition of β (1-3) β (1-4)
Glucan in Different Layers of Abdominal Skin

| | Mean Percent Deposition | | | | | |
|---|---|---|---|---|---|---|
| | COMPOSITION | | COMPOSITION | | | |
| | 1455 | | 1450 | | Control | |
| | Per- | Standard | Per- | Standard | Per- | Standard |
| | cent | Deviation | cent | Deviation | cent | Deviation |
| Medium | — | — | — | — | — | — |
| Swab | — | — | — | — | — | — |
| Horny | 8.7 | 1.2 | 12.8 | 1.9 | 0.6 | 0.2 |
| layer | | | | | | |
| Epidermis | 5.9 | 1.3 | 11.6 | 2.0 | 0.8 | 0.2 |
| Dermis | 2.4 | 0.5 | 4.1 | 1.1 | 0.6 | 0.1 |
| Subcutis | 1.4 | 0.5 | 1.5 | 0.4 | 0.9 | 0.1 |

The documentation of the findings by photographs (not shown) also demonstrated a significant uptake of the β-glucan into the epidermis layer of the skin samples.

The measurement of fluorescence was performed in accordance with quality control procedures and documentations. Control numbers of the BACTIDROP™ Calcofluor White were tested using recognized quality control organisms and were found to be acceptable.(Microbiology M. Pettenkofer Institute, München). Statistical evaluation was carried out by the statistics software package SAS/STATISTICA®. Both the hardware and the software used were validated.

### Example 13

### Pharmaceutical Compositions for Treating or Preventing a Skin Condition, an Inflammation, an Irritation or an Allergy Associated with an Ectoparasitic Infection or Infestation on an Animal

Composition A: To 100 ml of CEAPRO Certified Organic™ (50 ppm certified organic avenanthramide extract) (50 ppm avenanthramides in water (40%) and 1-3 propylene glycol (60%)) are added 150 ml of ethanol with stirring to provide a 20 ppm avenanthramide solution.

Composition B: To Imidacloprid (1000 gm) in 10000 gm of a solvent comprising ethyl lactate (5000 gm) and benzyl alcohol (5000 gm) was added with stirring 267 mL of a 750 ppm solution of avenanthramides in 50% ethanol.

Composition C: Two Litres of Ceapro Inc.'s 100 ppm avenanthramide extract was reduced under vacuum to a residue or lyophilized to a powder. The residue or powder produced was then added with stirring to a solution of Imidacloprid (1000 gm) in 10000 gm of a solvent mixture comprising ethyl lactate (5000 gm) and benzyl alcohol (5000 gm).

All three compositions can be applied to a companion animal as a topical spot/drop for treating or preventing a skin condition, an inflammation, an irritation or an allergy (such as flea allergy dermatitis) associated with an ectoparasitic infection or infestation on an animal. The third composition, which includes imidacloprid, can kill adult fleas.

One or more currently preferred embodiments have been described by way of example. It will be apparent to persons skilled in the art that a number of variations and modifications can be made without departing from the scope of the invention as defined in the claims.

What is claimed is:

1. A method for treating or preventing a skin condition, an inflammation, an irritation or an allergy associated with an ectoparasitic infection or infestation on an animal, comprising applying to the skin of the animal a pharmaceutical composition comprising a therapeutically effective amount of one or more than one avenanthramide and a pharmaceutically acceptable diluent or carrier.

2. The method according to claim 1, wherein the pharmaceutical composition further comprises a therapeutically effective amount of an ectoparasiticidal agent.

3. The method according to claim 2, wherein the ectoparasiticidal agent is an insecticide.

4. The method according to claim 1, wherein the method is for treating or preventing flea allergy dermatitis on the animal.

5. The method according to claim 2, wherein the pharmaceutical composition further comprises a therapeutically effective amount of an endoparasiticidal agent for treating or preventing an endoparasitic infection or infestation in the animal.

6. The method according to claim 5, wherein the endoparasiticidal agent is an anti-helminthic agent.

7. The method according to claim 1, wherein the pharmaceutical composition further comprises a therapeutically effective amount of a cereal β-glucan and/or one or more than one anti-inflammatory agent.

8. The method according to claim 2, wherein the pharmaceutical composition further comprises a therapeutically effective amount of a cereal β-glucan and/or one or more than one anti-inflammatory agent.

9. The method according to claim 5, wherein the pharmaceutical composition further comprises a therapeutically effective amount of a cereal β-glucan and/or one or more than one anti-inflammatory agent.

10. The method according to claim 1, wherein the one or more than one avenanthramide is present at a concentration of between about 0.0001 and about 375 ppm or any value or subrange therebetween.

11. The method according to claim 1, wherein the one or more than one avenanthramide is present at a concentration of between about 0.3 and about 15 ppm or any value or subrange therebetween.

12. The method according to claim 1, wherein the one or more than one avenanthramide is present in a concentration of between about 1.5 and about 4.5 ppm or any value or subrange therebetween.

13. The method according to claim 1, wherein the pharmaceutical composition comprises between 0.1 and 25% weight percent of an oat extract comprising the one or more than one avenanthramide at a concentration of between about 1 and about 1500 ppm or any value or subrange therebetween, based on the oat extract.

14. The method according to claim 13, wherein the oat extract comprises the one or more than one avenanthramide at a concentration of between about 3 and about 45 ppm or any value or subrange therebetween, based on the oat extract.

15. The method according to claim 1, wherein the pharmaceutical composition is the in the form of a solution, gel, lotion, cream, ointment or powder.

16. The method according to claim 1, wherein the pharmaceutical composition is in the form of a solution applied to the animal as a spray or as a spot.

17. The method according to claim 1, wherein the one, or more than one avenanthramide of the composition is produced by a process comprising the following steps:
a. Milling whole oats,
b. Extracting the resulting oatmeal or milled oat fraction with a solvent,
c. Adjusting the pH of the resulting oat extract to <4.0,
d. Membrane filtration of the oat extract with a pH <4.0 through a membrane <10⁴ MWCO.

18. A therapeutic pharmaceutical composition for treating or preventing a skin condition, an inflammation, an irritation or an allergy associated with an ectoparasitic infection or infestation on an animal, comprising a therapeutically effective amount of one or more than one avenanthramide and a pharmaceutically acceptable diluent or carrier.

19. The pharmaceutical composition according to claim 18, further comprising a therapeutically effective amount of a cereal β glucan and/or one, or more than one anti-inflammatory agent.

20. A therapeutic/parasiticidal pharmaceutical composition for treating or preventing a skin condition, an inflammation, an irritation or an allergy associated with an ectoparasitic infection or infestation on an animal, comprising a therapeutically effective amount of one or more than one avenanthramide, a therapeutically effective amount of an ectoparasiticidal agent and a pharmaceutically acceptable diluent or carrier.

21. The pharmaceutical composition according to claim 20, further comprising a therapeutically effective amount of a cereal β glucan and/or one, or more than one anti-inflammatory agent.

22. A therapeutic/parasiticidal pharmaceutical composition for treating or preventing a skin condition, an inflammation, an irritation or an allergy associated with an ectoparasitic infection or infestation on an animal and an endoparasitic infection or infestation in the animal, comprising a therapeutically effective amount of one or more than one avenanthramide, a therapeutically affective amount of an ectoparasiticidal agent, a therapeutically effective amount of endoparasiticidal agent and a pharmaceutically acceptable diluent or carrier.

23. The pharmaceutical composition according to claim 22, further comprising a therapeutically effective amount of a cereal β glucan and/or one, or more than one anti-inflammatory agent.

24. A therapeutic/parasiticidal pharmaceutical combination for treating or preventing a skin condition (such as erythema or pruritus), an inflammation (e.g. otitis), an irritation or an allergy associated with an ectoparasitic infection or infestation on an animal, comprising a first pharmaceutical composition comprising a therapeutically effective amount of one or more than one avenanthramide and a pharmaceutically acceptable diluent or carrier, and a second pharmaceutical composition comprising a therapeutically affective amount of an ectoparasiticidal agent and a pharmaceutically acceptable diluent or carrier, wherein the first and second pharmaceutical compositions are for separate, sequential or simultaneous administration.

25. The pharmaceutical combination according to claim 24, further comprising a third pharmaceutical composition for treating or preventing an endoparasitic infection or infestation in the animal, the third pharmaceutical composition comprising a therapeutically effective amount of endoparasiticidal agent and a pharmaceutically acceptable diluent or carrier, wherein the third pharmaceutical composition is for separate, sequential or simultaneous administration with the first and the second compostions.

26. A use of a pharmaceutical composition comprising a therapeutically effective amount of one or more than one avenanthramide and a pharmaceutically acceptable diluent or carrier, for treating or preventing a skin condition, an inflammation, an irritation or an allergy associated with an ectoparasitic infection or infestation on an animal.

27. The use according to claim 26, wherein the pharmaceutical composition further comprises a therapeutically effective amount of an ectoparasiticidal agent.

28. The use according to claim 27, wherein the ectoparasiticidal agent is an insecticide.

29. The use according to claim 26, wherein the pharmaceutical composition is for treating or preventing flea allergy dermatitis on the animal.

30. The use according to claim 27, wherein the pharmaceutical composition further comprises a therapeutically effective amount of an endoparasiticidal agent for treating or preventing an endoparasitic infection or infestation in the animal.

31. The use according to claim 30, wherein the endoparasiticidal agent is an anti-helminthic agent.

32. The use according to claim 26, wherein the pharmaceutical composition further comprises a therapeutically effective amount of a cereal β-glucan and/or one or more than one anti-inflammatory agent.

33. The use according to claim 27, wherein the pharmaceutical composition further comprises a therapeutically effective amount of a cereal β-glucan and/or one or more than one anti-inflammatory agent.

34. The use according to claim 30, wherein the pharmaceutical composition further comprises a therapeutically effective amount of a cereal β-glucan and/or one or more than one anti-inflammatory agent.

35. The use according to claim 26, wherein the one or more than one avenanthramide is present at a concentration of between about 0.0001 and about 375 ppm or any value or subrange therebetween.

36. The use according to claim 26, wherein the one or more than one avenanthramide is present at a concentration of between about 0.3 and about 15 ppm or any value or subrange therebetween.

37. The use according to claim 26, wherein the one or more than one avenanthramide is present in a concentration of between about 1.5 and about 4.5 ppm or any value or subrange therebetween.

38. The use according to claim 1, wherein the pharmaceutical composition comprises between 0.1 and 25% weight percent of an oat extract comprising the one or more than one avenanthramide at a concentration of between about 1 and about 1500 ppm or any value or subrange therebetween, based on the oat extract.

39. The use according to claim 38, wherein the oat extract comprises the one or more than one avenanthramide at a concentration of between about 3 and about 45 ppm or any value or subrange therebetween, based on the oat extract.

40. The use according to claim 26, wherein the pharmaceutical composition is the in the form of a solution, gel, lotion, cream, ointment or powder.

41. The use according to claim 26, wherein the pharmaceutical composition is in the form of a solution applied to the animal as a spray or as a spot.

42. The use according to claim 26, wherein the one, or more than one avenanthramide of the composition is produced by a process comprising the following steps:
a. Milling whole oats,
b. Extracting the resulting oatmeal or milled oat fraction with a solvent,
c. Adjusting the pH of the resulting oat extract to <4.0,
d. Membrane filtration of the oat extract with a pH <4.0 through a membrane <10⁴ MWCO.
